(19) United States
(12) Patent Application Publication
(10) Pub. No.: US 2007/0066979 A1
(43) Pub. Date: Mar. 22, 2007

(54) Epilator having a long-hair cutter

(75) Inventor: Ali Beytollahi, GRAZ (AT)

Correspondence Address: CHAPMAN AND CUTLER, 111 WEST MONROE STREET, CHICAGO, IL, 60603, US

(73) Assignee: Payer Internetional Technologies GmbH & Co. KG, St. Bartholoma (AT)

(21) Appl. No.: 10/562,774

(22) PCT Filed: Jul. 1, 2004
(86) PCT No.: PCT/AT04/00235

§ 371 Date: Dec. 29, 2005

(30) Foreign Application Priority Data
Jul. 1, 2003 (AT) GM 460/2003

(51) Int. Cl. A61B 17/50

(52) U.S. Cl. 606/133

(57) ABSTRACT

In an epilation device (1) including an epilation head (3) having clamping means for clamping and plucking hairs, wherein a long-hair cutter is arranged next to the epilation head (3), a protective comb (4) capable of being put on and off the epilation head (3) is provided to cover the cutting blades (32) of the long-hair cutter (29).

The invention relates to an epilation device including an epilation head having clamping means for clamping and plucking hairs, wherein a long-hair cutter is arranged next to the epilation head.

Epilation devices comprising a long-hair cutter in addition to an epilation head have already been known from the prior art. JP 4-348 704, for instance, discloses an epilation device whose head frame houses an epilation cylinder as well as a long-hair cutter including two cutting blades mounted to be movable relative to each other. By epilation cylinder, the total of all clamping elements which rotate about a common axis and are pressed against one another in the region of skin contact to clamp and pluck the hairs is usually referred to in such a case. As far as JP 4-348 704 is concerned, the clamping elements are formed by rotating discs which are alternately brought into mutual contact in the region of the plucking zone. Devices comprising a cutter unit in addition to an epilation unit in most cases will also require a drive for the oscillating movement of the cutting blades of the long-hair cutter in addition to the rotary drive. To this end, an obliquely arranged disc is connected with the axis of rotation of the epilation cylinder in the configuration according to JP 4-348 704, which disc engages, by its circumference, a driver rigidly connected with a cutting blade of the long-hair cutting system. In this manner, kind of a wobble plate is created, on which the oscillating movement for the cutting blade can be tapped. Such a drive, however, involves a number of problems. Thus, it is, for instance, impossible to set the long-hair cutter out of operation or arrange the same in a height-adjustable manner. In the configuration according to JP 4-348 704, only the simultaneous operation of the long-hair cutter and the epilation system is, hence, feasible and, therefore, only the combined use of the two systems is conceivable.

Furthermore, a hair-removal device has become known from WO 01/97748, on one side of which an epilation head and on the other side of which a cutting or shearing head are arranged. The two systems can, thus, be used independently of each other, whereby it is also feasible to separately switch on and off each one of the two systems. The device known from WO 01/97748, however, involves the drawback that a combined application, i.e., the simultaneous use of the epilation system and the long-hair cutter, is not possible.

Similar drawbacks will be encountered with the hair-removal device according to EP 630 596 A1. There, a main device including two mutually exchangeable add-on parts capable of being put on and off the main device is proposed, one of the said add-on parts being designed as an epilation head and the other one of said add-on parts being designed as a cutting means. Again, only the alternative use of the two systems is feasible in that case too.

The present invention aims to provide a device as versatile as possible, which enables the separate realization of both a hair shortening and hair removal, i.e., epilation, yet also permits the simultaneous application of the two systems. It has been shown in practice that an epilation procedure performed after an extended time interval following the epilation procedure carried out last, so that the hairs to be plucked have already exceeded a certain length, is extremely inefficient and, moreover, painful. The prerequisite for the safe and efficient action of the clamping elements of an epilation device is that the hairs to be plucked do not exceed a certain length. In this respect, it has been shown that a hair length of about 5 mm yields optimum results. It is thereby safeguarded that the clamping elements of the epilation device are able to seize the hairs close to the hairline so as to pluck the hairs at once. With longer hairs, however, the hairs to be plucked are more likely to be seized near their ends, whereby, in that case, an effective epilation will not be safeguarded, and great pain will be caused in addition, on the one hand, due to the elasticity of the hair itself and, on the other hand, due to the fact that a tension of the hair is only reached after a further rotation of the epilation cylinder about a defined angle.

To solve this object, the invention, departing from an epilation device of the initially defined kind, essentially consists in that a protective comb capable of being put on and off the epilation head is provided to cover the cutting blades of the long-hair cutter. Such a protective comb fulfils at the same time a plurality of functions. By the protective comb being capable of being put on the epilation head, a cover for the epilation system is provided such that the rotating clamping elements cannot enter into action. Thus, the sole use of the long-hair cutter is enabled without any hairs unintentionally reaching the epilation head and being seized by the same. When using the long-hair cutter, the protective comb covering the cutting blades of the long-hair cutter will slide over the skin surface of the user, thus enabling the hairs to be gently cut without causing any injuries. The protective comb also ensures the observance of a constant cutting height so as to maintain a cutting height that is particularly favorable for the subsequent epilation procedure. The mode of operation of the epilation device with the protective comb placed on the epilation head, thus, serves to precut the hairs that are to be epilated subsequently, whereby, as already pointed out above, pre-cutting to approximately 5 mm is of particular advantage to ensure gentle epilation. The long-hair cutter may, however, also be used to cut hairs in body regions where subsequent epilation is not intended. In any event, the arrangement of the protective comb according to the invention substantially enhances the variability of the combined epilation and cutting device, whereby switching from one mode of operation to another is feasible by simply putting on and off the protective comb.

In order to facilitate the sole use as an epilation device, the configuration is preferably devised such that the long-hair cutter is arranged to be extensible from an out-of-operation position into an operation position or, vice versa, retractable from an operation position into an out-of-operation position. By such a configuration, the long-hair cutter will merely be extended into the operation position if it is actually used, i.e., for instance, if the protective comb has been put on. When removing the protective comb, the clamping means of the epilation head are uncovered, and the epilation procedure should not be affected by the presence of the long-hair cutter unless it is required. To this end, the long-hair cutter is retracted into an out-of-operation position so as to enable the epilation device to be employed in a conventional manner.

If, however, a combined use of the long-hair cutter and the epilation head is desired, the long-hair cutter may be extended to enable in a single operation pre-cutting and the subsequent epilation procedure. In an advantageous manner, the configuration is further devised such that the long-hair cutter is designed to be height-adjustable for the adjustment of the cutting length. This enables the adjustment of the pre-cutting of the hair to any desired length, whereby the cutting length is defined by the distance of the cutting blades from the protective comb to be put onto the epilation head. According to a preferred further development, the long-hair cutter may be mounted in a resiliently floating manner. This enables particularly gentle cutting of the hair while allowing the adaptation of its contour to the skin surface. Such a configuration will be of advantage if also the protective comb is resiliently mounted, whereby a constant cutting length will be kept by the appropriate coupling of the resilient movement of the protective comb with that of the long-hair cutter.

In devices for use both as an epilation device and as a long-hair cutter, both a rotating drive for the epilation head and an oscillating drive for the cutting blade of the long-hair cutter must be provided. In this respect, also the different driving speeds are to be taken into account, since the rotation frequency of the epilation cylinder will, as a rule, lie somewhat below the oscillation frequency of the cutting blades. It is, hence, necessary to provide two drives having different transmissions and gear reductions, wherein both a rotary and a translational drive are to be accomplished by a single motor in order to comply with the limited space conditions and energy reserves. To this end, the configuration is preferably further devised such that a double-armed rocker pivotable about a rotation axis extending parallel with the longitudinal axis of the device is provided, whereby the free end of one arm carries a preferably spherically shaped driver for the oscillating drive of the long-hair cutter and the free end of the other arm carries a coupling member which converts the rotation of a pinion driven by the motor into an oscillatory movement. The two-armed rocker, thus, serves to tap the rotational movement delivered by the motor and to pass it on to the long-hair cutter as a translational oscillatory movement. The driver provided for this purpose on the free end of said one arm may preferably be spherically shaped, engaging in a fork-shaped projection of a carrier connected with one of the two cutting blades. The fork-shaped projection is arranged in a manner that, in a configuration in which the long-hair cutter is arranged to be adjustable in the height direction, will only cooperate with the driver of the rocker if the long-hair cutter has been extended into the operation position. This causes the long-hair cutter to be driven only if actually in use, which helps to save energy.

The conversion of the rotational movement of the motor or respective pinion into an oscillatory movement for the cutting blade of the long-hair cutter is preferably effected in that the coupling member comprises a groove extending parallel with the longitudinal axis and engaged by a driver pin eccentrically arranged on the pinion.

The drives of epilation devices, as a rule, are realized by electromotors exhibiting speeds of between 10,000 and 12,000 revolutions per minute. The configuration, therefore, is advantageously further devised such that the pinion cooperating with the coupling member of the rocker is coupled to the drive motor via an at least single-stage reduction gear. For driving an epilation cylinder housed within the epilation head and carrying the clamping means, the former is preferably coupled with the drive motor via a further reduction gear, said reduction gear for the drive of the epilation cylinder having a higher gear reduction than that provided for the oscillatory drive of the long-hair cutter. It is, thus, taken into account that the epilation cylinder is to rotate at a frequency lying below the respective frequency of the oscillating movement of the long-hair cutter.

In order to achieve an even higher variability of the device according to the invention, the configuration may be further devised such that the epilation head is designed to be detachable, with coupling members being provided to receive a shearing head. In such a configuration, the epilation head is designed as an easy-change head and can be replaced with a shearing head after complete removal so as to provide a conventional dry shaver by the combination of a shearing system with the long-hair cutter.

In the following, the invention will be explained in more detail by way of an exemplary embodiment schematically illustrated in the drawing. Therein,

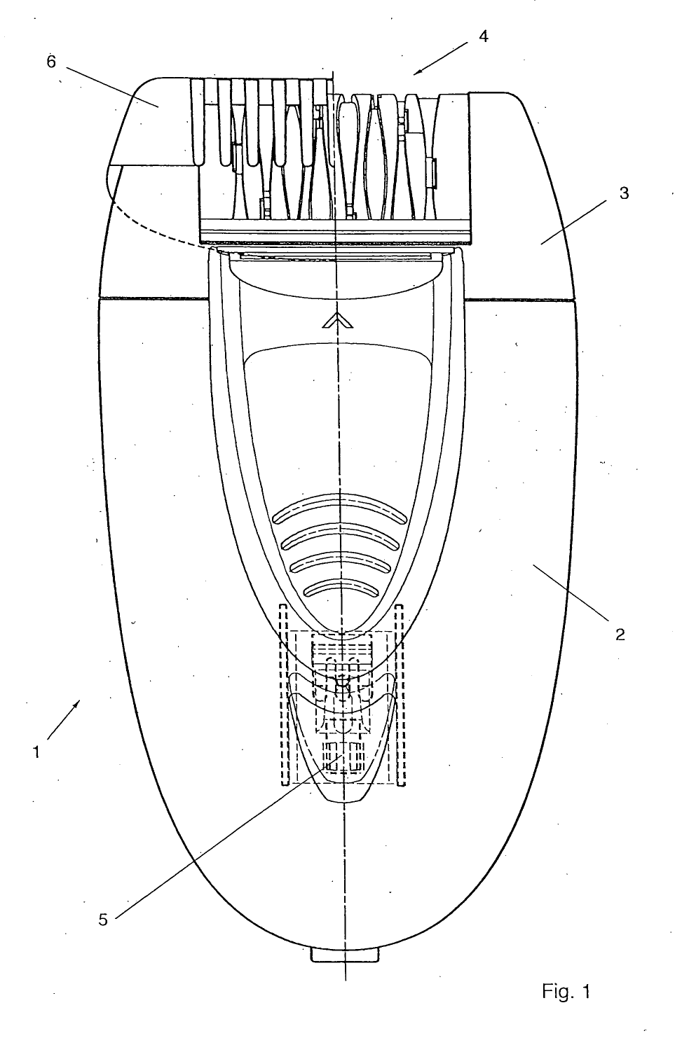

FIG. 1 is a front view of an epilation device,

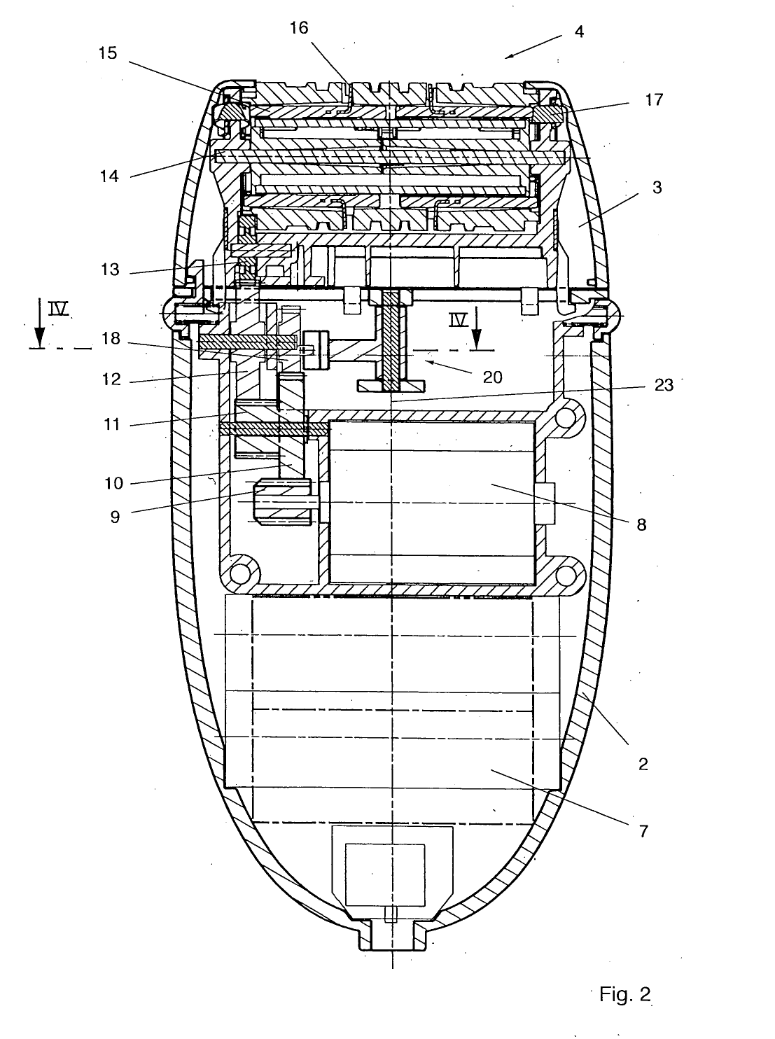

FIG. 2 is a first sectional view,

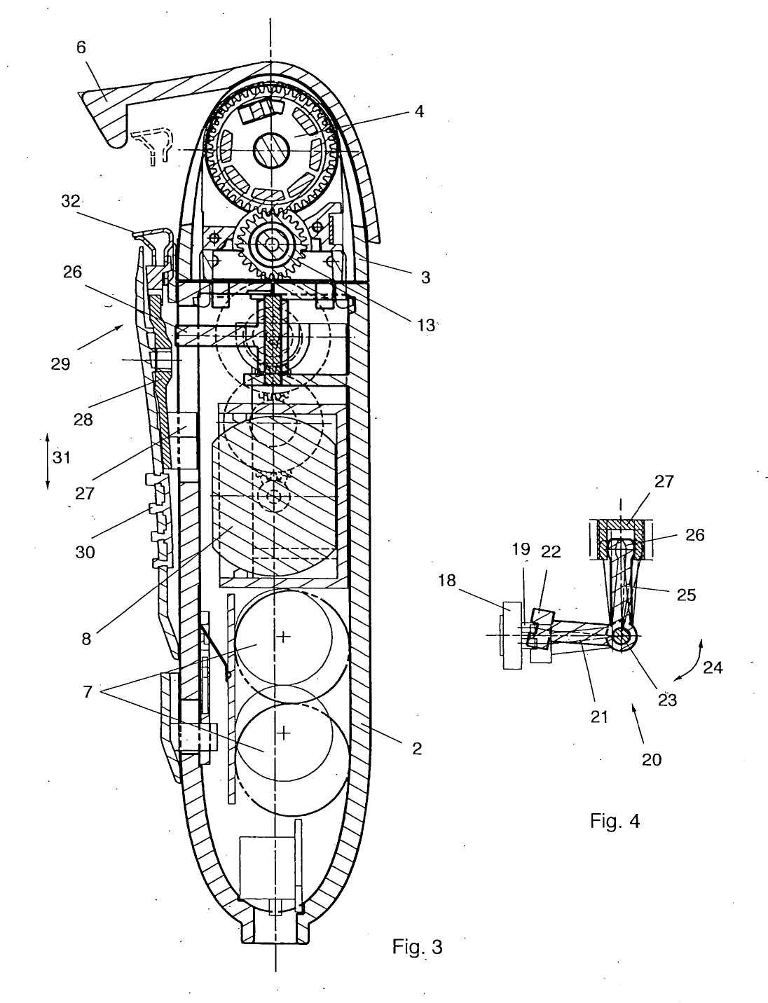

FIG. 3 is a second sectional view, and

FIG. 4 is a detailed view of the rocker, sectioned along line IV-IV of FIG. 2.

FIG. 1 depicts an epilation device 1 comprising a main body 2 and an epilation head 3. In the region of the opening of the head frame, the epilation cylinder 4 is to be seen, which comprises a plurality of clamping elements that serve to clamp and pluck hairs. The switch for switching the epilation device on and off is denoted by 5. In the illustration on the left-hand side of FIG. 1, the epilation device 1 is shown with an attached comb 6, and it is apparent that the epilation cylinder 4 is covered with the comb 6 put on, so that no hairs may enter the region of the rotating clamping means.

From the sectional illustration according to FIG. 2, it is apparent that an energy supply unit 7 and an electric drive motor 8 are arranged in the interior of the main housing 2. The driven pinion 9 of the motor 8 engages in a pinion 10 which is rigidly connected with a further pinion 11, whereby driving of the rotation cylinder 4 about the axis 14 is effected via pinions 12 and 13. The rotation cylinder comprises a plurality of supports 15 for clamping elements 16 which are actuated by pinch rollers 17 in a manner that the hairs to be plucked will be seized by the clamping elements when passing the plucking zone.

The driving of the long-hair cutter, which is arranged on the outer side of the main body 2 as can best be seen in FIG. 3, is effected from the pinion 10. The pinion 10 engages with a pinion 18 including an eccentrically arranged driver pin 19 which cooperates with a coupling part 22 arranged on the free end of an arm 21 of a rocker 20. The rocker 20 is mounted to be rotatable about an axis 23, a detailed view of the rocker 20, sectioned along line IV-IV of FIG. 2, being illustrated in FIG. 4. It will be understood that the rocker 20 is caused to carry out an oscillating rotational movement in the sense of arrow 24 by the driver pin 19 engaging in a groove of the coupling part 22. The second arm 25 of the rocker 20 carries on its free end a spherically shaped driver part 26, which engages in a respective, fork-shaped projection 27 connected with a carrier 28 for a cutting blade of the long-hair cutter 29.

From the sectional illustration according to FIG. 3, the long-hair cutter 29 is more clearly apparent, said long-hair cutter 29 being adjustable in the sense of double arrow 31 via a grip end 30 and said driver end piece 26 engaging in the fork-shaped projection 27 in the extended position, i.e., in the position of the cutting blades 32 illustrated in broken lines. In the extended position of the long-hair cutter, the cutting blades 32 come to lie at a predetermined distance relative to the protective comb 6, thus defining a constant hair cutting length. From the illustration according to FIG. 3, it is again clearly apparent that the protective comb 6 covers the epilation head 3 while, at the same time, offering sufficient protection from the sharp-edged cutting blades 32 and facilitating the guidance of the device over the skin surface.

1. An epilation device, comprising an epilation head comprising clamping means for clamping and plucking hairs, wherein a longhair cutter is arranged next to the epilation head, and a protective comb capable of being put on and off the epilation head (3) is provided to cover cutting blades (32) of the long-hair cutter (29).

2. An epilation device according to claim 1, wherein the long-hair cutter (29) is arranged to be extensible from an out-of-operation position into an operation position.

3. An epilation device according to claim 1, wherein the long-hair cutter (29) is designed to be height adjustable for adjustment of cutting length.

4. An epilation device according to claim 1, wherein the long-hair cutter (29) is mounted in a resiliently floating manner.

5. An epilation device according to claim 1, further comprising a double-armed rocker (20) pivotable about a rotation axis (23) extending parallel with a longitudinal axis of the device, whereby a free end of one arm (25) of the rocker (20) carries a driver (26) for an oscillating drive of the long-hair cutter (29) and a free end of a second arm (21) of the rocker (20) carries a coupling member (22) which converts the rotation of a pinion (18) driven by a drive motor (8) into an oscillatory movement.

6. An epilation device according to claim 5, wherein the coupling member (22) comprises a groove extending parallel with the longitudinal axis and engaged by a driver pin (19) eccentrically arranged on the pinion (18).

7. An epilation device according to claim 5, wherein the pinion (18) cooperating with the coupling member (22) of the rocker (20) is coupled to the drive motor (8) via an at least single-stage reduction gear.

8. An epilation device according to claim 1, wherein the epilation head (3) comprises at least one epilation cylinder (4) comprising said clamping means, said epilation cylinder being coupled with a drive motor (8) via a reduction gear and drivable for rotation.

9. An epilation device according to claim 8, wherein the reduction gear for the drive of the epilation cylinder (4) has a higher reduction than that provided for oscillatory drive of the long-hair cutter (29).

10. An epilation device according to claim 1, wherein the epilation head (3) is designed to be detachable, and comprises coupling members to receive a shearing head.

11. An epilation device according to claim 2, wherein the driver (26) is spherically shaped.

12. An epilation device according to claim 2, wherein the long-hair cutter (29) is designed to be height adjustable for adjustment of cutting length.

13. An epilation device according to claim 2, wherein the long-hair cutter (29) is mounted in a resiliently floating manner.

14. An epilation device according to claim 3, wherein the long-hair cutter (29) is mounted in a resiliently floating manner.

15. An epilation device according to claim 2, further comprising a double-armed rocker (20) pivotable about a rotation axis (23) extending parallel with a longitudinal axis of the device, whereby a free end of one arm (25) of the rocker (20) carries a driver (26) for an oscillating drive of the long-hair cutter (29) and a free end of a second arm (21) of the rocker (20) carries a coupling member (22) which converts rotation of a pinion (18) driven by a drive motor (8) into an oscillatory movement.

16. An epilation device according to claim 3, further comprising a double-armed rocker (20) pivotable about a rotation axis (23) extending parallel with a longitudinal axis of the device, whereby a free end of one arm (25) of the rocker (20) carries a driver (26) for an oscillating drive of the long-hair cutter (29) and a free end of a second arm (21) of the rocker (20) carries a coupling member (22) which converts rotation of a pinion (18) driven by a drive motor (8) into an oscillatory movement.

17. An epilation device according to claim 4, further comprising a double-armed rocker (20) pivotable about a rotation axis (23) extending parallel with a longitudinal axis of the device, whereby a free end of one arm (25) of the rocker (20) carries a driver (26) for an oscillating drive of the long-hair cutter (29) and a free end of a second arm (21) of the rocker (20) carries a coupling member (22) which converts rotation of a pinion (18) driven by a drive motor (8) into an oscillatory movement.

18. An epilation device according to claim 6, wherein the pinion (18) cooperating with the coupling member (22) of the rocker (20) is coupled to the drive motor (8) via an at least single-stage reduction gear.

19. An epilation device according to claim 2, wherein the epilation head (3) comprises at least one epilation cylinder (4) comprising said clamping means, said epilation cylinder being coupled with a drive motor (8) via a reduction gear and drivable for rotation.

20. An epilation device according to claim 2, wherein the epilation head (3) is designed to be detachable, comprising coupling members to receive a shearing head.
